(19) United States
(12) Patent Application Publication
(10) Pub. No.: US 2004/0107965 A1
(43) Pub. Date: Jun. 10, 2004

(54) System and method for monitoring gas supply and delivering gas to a patient

(76) Inventors: Randall S. Hickle, Lubbock, TX (US); Samsun Lampotang, Gainesville, FL (US); Paul J. Niklewski, Cincinnati, OH (US); Gregory D. Bishop, Hamilton, OH (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/660,286

(22) Filed: Sep. 11, 2003

Related U.S. Application Data
(60) Provisional application No. 60/410,963, filed on Sep. 16, 2002.

(51) Int. Cl. A61M 16/00

(52) U.S. Cl. 128/204.22

(57) ABSTRACT

A gas supply and monitoring system is provided for use in many medical system which can include, but is not limited to, a sedation and analgesia system, anesthesia machines, dental gas systems, and veterinary systems which deliver gasses. The gas supply and monitoring system can include a gas source; a variable size orifice system connected to the gas source wherein the variable size orifice system has a gas output supply opposite of the gas source; and a sensor system connected to the gas output supply wherein the sensor system can be used to verify a patient receives the appropriate gas output supply.

### CROSS REFERENCE TO RELATED APPLICATIONS

This application claims the benefit and priority from U.S. provisional application, Serial No. 60/410963, filed on Sep. 16, 2002, which is incorporated by reference herein in its entirety. The present application cross references and incorporates by reference copending U.S. Ser. No. U.S. Ser. No. 09/324,759, filed Jun. 3, 1999, U.S. Ser. No. 09/592,943, filed Jun. 13, 2000, and U.S. Ser. No. 09/878,922, filed Jun. 13, 2001.

### FIELD OF THE INVENTION

The present invention relates, in general, to gas delivery and monitoring systems and, more particularly, to gas delivery and monitoring systems associated with medical devices

### BACKGROUND OF THE INVENTION

Typically, large medical facilities such as hospitals and outpatient surgery centers rely on in-house oxygen delivery from fixed, central locations within the facility. The distant nature of central gas supplies, causes a clinician performing a medical procedure to be able to only verify that a proper gas inlet, such as, for example, an oxygen hose or inlet, is properly hooked up to its corresponding gas outlet. The distant nature does not allow the clinician to verify that a proper gas inlet is properly hooked up to a proper gas source. There is no practical and expedient means for a clinician to trace gas piping from a central gas outlet all the way back to an actual gas source. In many environments, tanks, or pipes containing hypoxic gases such as, for example, nitrous oxide, are housed or routed in close proximity to tanks or pipes containing oxygen. In such environments, there have been occurrences of patient injury and even deaths resulting from hookup of an improper hypoxic gas source to an oxygen delivery outlet.

Similar potentially life-threatening episodes can occur in circumstances where tanks are either misconnected, mislabeled, misfilled, or misidentified. An anesthesia, sedation, and/or analgesia system may carry nitrous oxide (N₂O) and oxygen (O₂) cylinders simultaneously, where accidental misconnection of an N₂O cylinder to an O₂ cylinder yoke is potentially fatal. In response to a need to guard against improper oxygen delivery during medical procedures, a number of devices and schemes have been developed in attempts to improve patient safety including color coding of gas cylinders and gas hoses, the DISS (Diameter Index Safety System) for gas hoses and the PISS (Pin Index Safety System) for cylinder post valves and yokes. Despite these safety features, gas mix-ups continue to happen and unnecessarily claim lives of patients due to human error and due to the fact that current safety measures are not foolproof.

One technique for measuring oxygen in an external environment is a galvanic cell oxygen sensor, also known as a fuel cell, where positive and negative electrodes (an anode and a cathode) are placed in a liquid electrolyte bath. The potential difference between the electrodes is proportional to a partial pressure of oxygen that diffuses into the fuel cell via an oxygen-permeable membrane. Such sensors are capable of measuring oxygen concentrations near room temperature and are common in medical and environmental applications. A drawback of these sensors is that an oxide layer may build up on the cell of the sensor during prolonged, for example, more than 5 minutes disconnection and exposure to oxygen which may happen, among other times, during transport, storage, removal from an airtight (to minimize exposure to O₂ in room air) package and temporary or accidental disconnection of the O₂ sensor that may limit the output of the galvanic cell and interfere with accurate measurement of O₂. The oxide layer may be removed by connecting the O₂ sensor to its monitor for a period of time similar to the time of disconnection or the amount of time the sensor has been removed from its airtight packaging, up to a maximum of approximately 24 hours. Therefore, after reconnecting a used sensor after prolonged disconnection or upon removal of a new sensor from a sealed package, galvanic cell oxygen sensors may need to be connected for up to 24 hours before reading correctly.

Although liquid electrolyte oxygen sensors work at ambient temperatures, such sensors have numerous problems. The chemical reaction of the liquid electrolyte tends to run fairly quickly, limiting the total operational lifespan of the sensors. Moreover, the rate of reaction, which affects the potential difference between the electrodes, is a function of the concentration of the liquid electrolyte, and the concentration of the electrolyte changes as the reaction occurs over time. Further, the concentration of liquid electrolyte changes as it dries out over the length of its service life. This means that such oxygen sensors need to be regularly manually recalibrated, often on a daily basis, to account for the change in concentration of the electrolyte. Recalibration is often a time consuming and costly process. Though these sensors are highly effective for a period of time, compensation for the above problems drives up the cost of the sensors and reduces their effectiveness.

Paramagnetic sensors are typically used specifically for measuring oxygen concentration. The design of these sensors are based on oxygen's high degree (compared to other gasses) of sensitivity to magnetic forces. One such sensor design includes a symmetrical, two chambered cell with identical chambers for sample and reference gas (e.g., air) streams. The chambers are joined at an interface by a differential pressure transducer or microphone. Sample and reference gases are pumped through these chambers and a strong magnetic field surrounding the regions acts on oxygen molecules to generate a pressure difference between the two sides of the cell. The magnetic field causes the transducer to produce a voltage proportional to oxygen partial pressure. This device requires frequent calibration, is costly in and of itself, and depends on the availability of certain skills of its operator for proper operation.

Known systems that receive, generate, and/or deliver oxygen generally do not take pro-active steps to prevent a potentially harmful situation. For example, in the event that oxygen delivery from an oxygen delivery system becomes hypoxic (i.e., O₂ concentration below 20%) or anoxic (i.e., O₂ concentration of 0%), existing systems simply alert a clinician that oxygen levels have fallen below a predetermined level. In these circumstances, it is still necessary for the clinician to diagnose the problem and remedy the situation. Due to a vast number of alarms associated with existing oxygen delivery and/or anesthesia systems, it may be awhile before the clinician can diagnose and correct the problem. Depending on the species and concentration of gas being incorrectly administered, a time delay in correcting a hypoxic gas supply condition may have dire consequences.

Regarding integrated sedation and analgesia systems, where drug delivery is integrated with patient monitoring systems, a need for oxygen delivery is often a result of respiratory depression that may be caused by sedative and analgesic drugs. It may be crucial that a patient under the influence of such drugs receives an elevated concentration of oxygen to support proper gas exchange. Should an improper or hypoxic gas be delivered to such a patient, it would be advantageous to provide a system that delivers back up oxygen and/or room air while deactivating drug delivery to the patient.

A number of existing methods and systems have had moderate success in providing oxygen sensors with extended periods of useful life. Existing sensors are often designed for continuous monitoring of oxygen and/or other gases during a procedure to ensure that the composition of a gas mixture, influenced by multiple time varying parameters, remains at predetermined levels. To improve patient safety, among others, it must be verified that 1) at the beginning of a procedure, an oxygen supply is correctly attached to an oxygen delivery system, and 2) that oxygen concentration levels are appropriate if a patient experiences an oxygen desaturation event.

Most O₂ sensors are designed to produce a graded output such that the O₂ sensor can differentiate between, for example, 25% O₂ and 30% O₂. In applications of an O₂ sensor where the purpose of the sensor is to determine if gas supplied to an O₂ inlet of a medical system that delivers oxygen is really oxygen, there may be no need for a graded output from the O₂ sensor. The requirement from an O₂ sensor monitoring an O₂ supply is a real-time determination whether gas in an O₂ supply inlet is O₂ or not, with the O₂ sensor providing a binary output: yes or no. Eliminating the need for a graded output of O₂ concentration would remove the expense and additional hardware and software associated with providing accurate gas analysis over a desired measurement range of O₂.

### BRIEF SUMMARY OF THE INVENTION

The present invention provides oxygen delivery and O₂ supply monitoring systems and methods aimed at improving patient safety. The O₂ supply monitoring system is capable of detecting adverse or improper conditions in the supply of O₂ to a patient such as the O₂ supply system becoming hypoxic. The oxygen delivery system enhances patient safety by automatically deactivating delivery of improper or hypoxic gas in a timely manner upon the O₂ delivery system becoming hypoxic. The system may also activate either backup oxygen and/or room air delivery to a patient.

Certain embodiments of the system of the present invention may be used with an automated drug delivery system. In such embodiments, the system may deliver back up oxygen and/or room air while deactivating drug delivery to the patient should an improper or hypoxic gas be delivered to the patient.

The present invention further provides a system and method for sensing O₂ using any O₂ sensing means suitable for the purposes of ensuring patient safety. In embodiments of the present invention in which the O₂ sensor used is consumable, the system and method of the invention maximize the life of the sensor by minimizing the time periods where it is exposed to O2 and used. The present invention also provides a system utilizing an open-circuit, mask-free sedation and analgesia system, thereby substantially reducing the need for continuous monitoring of the concentration of oxygen delivered to a patient (because the spontaneously breathing patient has access to room air). Certain embodiments of the invention providing this sedation and analgesia system utilize an oxygen sensor that measures oxygen concentration solely at a beginning of procedures and during patient desaturation events, thereby reducing the use and prolonging the life of the oxygen sensor and reducing the manpower and expense of replacing used or depleted oxygen sensors.

Further embodiments of the system of the present invention utilize an oxygen sensor integral with a microprocessor, or other processing unit, that is capable of recalibrating itself, thereby reducing the need and expense for manual recalibration of the oxygen sensors.

The present invention also provides a system utilizing a binary O₂ sensor that is inexpensive and generally maintenance-free and does not require calibration and/or periodic replacement

### BRIEF DESCRIPTION OF THE FIGURES

FIG. 1 illustrates a block diagram of one embodiment of a gas delivery and monitoring system integral with a drug delivery system in accordance with the present invention.

FIG. 2 illustrates a detailed schematic of one embodiment of a gas delivery and monitoring system in accordance with the present invention.

FIG. 3 illustrates a flow chart of one embodiment of a method of operating a gas delivery and monitoring system in accordance with the present invention.

FIG. 4 illustrates a layout of a binary O₂ sensor of a type that exploits the relatively singular paramagnetic property of oxygen molecules to determine whether a gas flowing through the sensor is oxygen.

FIG. 5 illustrates an alternate embodiment which can replace variable size orifice valve of the gas delivery and monitoring system in accordance with the present invention.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

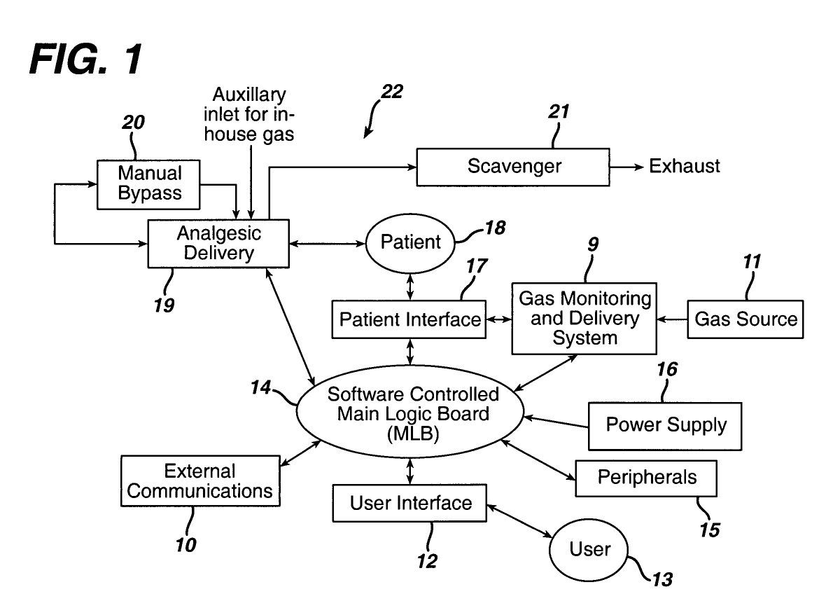

FIG. 1 illustrates a block diagram depicting one embodiment of the present invention comprising a sedation and analgesia system 22 having user interface 12, software controlled controller 14, peripherals 15, power supply 16, external communications 10, patient interface 17, scavenger 21, manual bypass 20, drug delivery system 19, gas source 11 and gas delivery system 9, where sedation and analgesia system 22 is operated by user 13 in order to provide sedation and/or analgesia to patient 18. Examples of sedation and analgesia system 22 that may be used with the invention, are disclosed and enabled by U.S. patent application Ser. No. 09/324,759, filed Jun. 3, 1999 which is herein incorporated by reference in its entirety. Examples of embodiments of patient interface 17 that may be used with the invention are disclosed and enabled by U.S. patent application Ser. No. 09/592,943, filed Jun. 13, 2000 and Ser. No. 09/878,922, filed Jun. 13, 2001 which are herein incorporated by reference in their entirety.

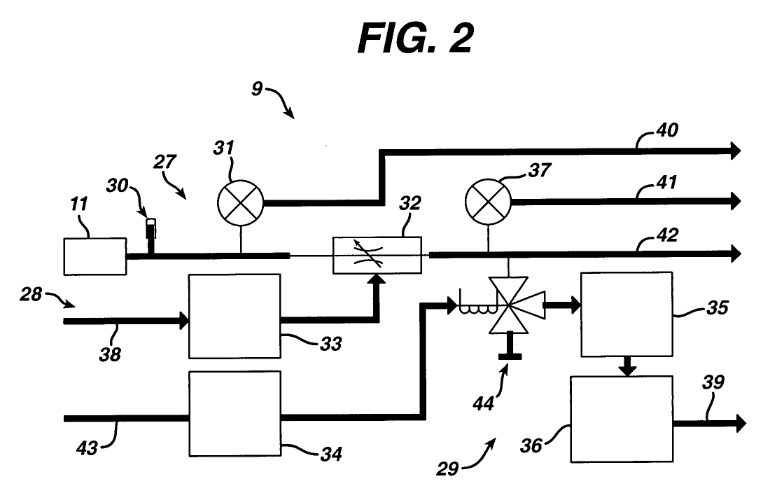

FIG. 2 illustrates a schematic depicting a more detailed view of one embodiment of gas monitoring and delivery system 9 and gas source 11 comprising variable size orifice system 27, which further comprises of pressure relief valve 30, high-side pressure sensor 31, high-side pressure output 40, variable size orifice valve 32, low-side pressure sensor 37, low-side pressure output 41, gas outflow 42. Gas monitoring and delivery system 9 further comprises of a control unit 28 which includes variable size orifice valve controller 33, variable size orifice valve control input 38, solenoid valve driver 34, control input 43 for sampling gas supplied to the patient. Gas monitoring and delivery system further includes a sensor system 29 which comprises solenoid-activated 2-way valve 44, gas sensor 35, gas sensor signal conditioner 36, and gas sensor output 39. Gas sensor 35 may be, for example, a Max-14 galvanic cell oxygen sensor from Maxtec, Inc. Gas source 11 may be an in-house gas supply, a portable gas supply, or any other suitable gas dispenser. Gas source 11 further comprises containment and delivery of oxygen, nitrous oxide, sedatives, analgesics, and/or other gases suitable for sedation and analgesia, deep sedation, general anesthesia or monitored anesthesia care or desirable combinations of suitable gases. Gas sensor 35 may be any sensor suitable for measuring oxygen such as, for example, galvanic or fuel cells, polarographic analyzers, paramagnetic analyzers, and/or magneto-acoustic analyzers. Examples of suitable sensors are disclosed by Dunigan in U.S. Pat. No. 6,099,707, Shen in U.S. Pat. No. 6,080,294, and Drzewiecki in U.S. Pat. No. 6,305,212.

Gas monitoring and delivery system 9 is, in one embodiment of the present invention, integral with a sedation and analgesia system 22. However, it is contemplated that gas monitoring and delivery system 9 may be used with any of a variety of medical systems to monitor and deliver gas to patient 18. The system 9 for monitoring and confirming the identity of a supplied gas is applicable to medical, dental and veterinary systems delivering oxygen and other medical gases such as sedation and analgesia delivery systems, anesthesia machines, anesthesia workstations, dental gas systems and analgesia equipment, and gas flow metering systems in human and veterinary fields. Pressure relief valve 30 may be any suitable pressure valve, such as, for example, model VRV-125B-N-75-X, made by GENERANT, where excessive gas pressure from gas source 11 may cause pressure relief valve 30 to purge gas resulting in decreased pressure. A pressure relief valve 30 may be located upstream from variable size orifice valve 32, downstream from variable size orifice valve 32, or in both locations. Placing pressure relief valve 30 downstream of variable size orifice valve 32 will release gas pressure in the event that kinks or occlusions occur in the tubing or hardware associated with gas monitoring and delivery system 9. Pressure relief valve 30 may be set to discharge gas at any threshold pressure such as, for example, 75 psig for an upstream pressure relief valve 30 and 25 psig for a downstream pressure relief valve 30. Gas monitoring and delivery system 9 may also incorporate a pressure regulator (not shown) in combination with, or in place of, pressure relief valve 30. A further embodiment of the present invention comprises completely closing variable size orifice valve 32 in the event that high-side pressure sensor 31 and/or low-side pressure sensor 37 detect excessive gas pressure. High-side pressure sensor 31 and/or low-side pressure sensor 37 may communicate with controller 14 digitally, whereby if an excessive pressure threshold is met in either high-side pressure sensor 31 or low-side pressure sensor 37, controller 14 will completely close variable size orifice valve 32, thereby interrupting gas delivery to patient 18.

High-side pressure sensor 31 may be any suitable gas pressure sensor such as, for example, the XCAL4100GN made by Honeywell. Low-Side pressure sensor 31 may be any suitable gas pressure sensor such as, for example, the XCAL430GN made by Honeywell. Gas outflow 42 to patient 18, in one embodiment of the present invention, is controlled in an open loop fashion using variable size orifice valve 32. Changing the amount of current flowing through the valve coil (not shown) of variable size orifice valve 32 varies the flow orifice of variable size orifice valve 32. An excessive gas pressure event detected by high-side pressure sensor 31 or low-side gas pressure sensor 37 may be transmitted digitally via high-side pressure output 40 or low-side pressure output 41, respectively, to controller 14. Controller 14, in one embodiment of the present invention, communicates with variable size orifice valve controller 33 via variable size orifice control input 38. Variable size orifice valve controller 33 may alter a flow orifice of variable size orifice valve 32 by varying current flow through a valve coil (not shown) as a result of communications received from controller 14. Varying the flow orifice of variable size orifice valve 32 causes changes in magnitude of an outflow of gas to patient 18. Other means of modulating flow rate or controlling flow such as, for example, pulse width modulation, voltage sensitive orifices, banks of on/off valves with each valve delivering twice as much flow as the valve with the next lower flow and on/off valves are also contemplated for use with the invention.

The present invention further comprises employing solenoid-activated 2-way valve 44, solenoid valve driver 34, gas sensor 35, and gas sensor signal conditioner 36 to determine concentration of O₂ for example, in gas outflow 42. In one embodiment of the present invention, solenoid-activated 2-way valve 44 is positioned downstream from variable size orifice valve 32; however, solenoid-activated 2-way valve 44 may be positioned at any suitable location within gas monitoring and delivery system 9, including upstream of variable size orifice valve 32. The present invention comprises controller 14 signaling solenoid valve driver 34, via gas sample control input 43, to enable solenoid-activated 2-way valve 44, thereby allowing a sample of gas to pass through solenoid-activated 2-way valve 44 to gas sensor 35. Controller 14 may initiate solenoid valve driver 34 to enable solenoid-activated 2-way valve 44 only during specified time periods. In one embodiment of the present invention, controller 14 signals solenoid valve driver 34 to enable solenoid-activated 2-way valve 44 solely at the beginning of a medical procedure or as a result of oxygen desaturation. Testing gas 42 at the beginning of a medical procedure informs user 13 that a proper gas, and optionally a proper concentration of gas, is connected to gas monitoring and delivery system 9. Enabling solenoid-activated 2-way valve 44 only at specified periods may prolong the life of gas sensor 35 by reducing the average time of use of gas sensor 35 during procedures. Enabling solenoid-activated 2-way valve 44 to allow gas sensor 35 to measure the concentration of gas 42 solely during critical monitoring periods may enhance patient safety while extending the useful life of gas sensor 35. The present invention comprises sampling the concentration of gas during initiation of gas monitoring and delivery system 9, in the event of a patient desaturation event, or at any other desirable time or untoward event. The present invention may further comprise a manual feature, where user 13 may initiate a gas concentration measurement at any time during a medical procedure.

Oxide film formation in a galvanic cell O₂ sensor upon disconnection and exposure to oxygen and its limiting effect on the cell output may interfere with the method of intermittently using an O₂ sensor to prolong the sensor's life. To prevent an oxide film from forming, a galvanic cell O₂ sensor used with the invention may always be left connected with active monitoring using hardware and/or software algorithms to verify that the galvanic cell O₂ sensor remains connected. For example, a galvanic cell output voltage of 0 may indicate that the galvanic cell is disconnected from its monitor or system. A timer may track the amount of time that the galvanic cell is disconnected. With this data about disconnection time, the control software may then require that the sensor if reconnected to the system must be allowed to stabilize for an amount of time similar to the disconnection time to prevent erroneous readings. The system may keep track whether the same sensor is being reconnected by means of a unique indicia associated with each O₂ sensor. In a further embodiment, the system of the present invention detects when a new O₂ sensor is inserted into the system and, where applicable, tracks a burn-in or warm-up period. The system may notify a user of unreliability of the O₂ sensor if the user attempts to initiate a procedure within the burn-in or warm-up period or the system may prevent initiation of a procedure altogether until the burn-in or warm-up period is completed or may only allow an O₂ sensor with a graded output, such as a galvanic cell, to be used in a gross binary mode, i.e., is it O₂ or not? Several means for the controller to detect the insertion of a new O₂ sensor into gas monitoring and delivery system 9 are contemplated for use with the present invention. The insertion of a new O₂ sensor may be detected by an abnormal output when the new O₂ sensor is exposed to room air or a calibration gas such as 100% O₂. Alternatively, a new O₂ sensor may be detected by reading a Quality Assurance Module (QAM) attached to the O₂ sensor or its package or wrapper. A QAM component and a system for reading a QAM component that may be used with the present invention are disclosed and enabled by U.S. patent application Ser. No. 60/310,227 filed Aug. 7, 2001 and Ser. No. 60/324,043 filed Sep. 24, 2001 which are herein incorporated by reference in their entirety.

Gas sensor 35 may be a galvanic or fuel cell, a polarographic sensor, a paramagnetic sensor, or any other suitable gas sensor. The present invention further comprises a plurality of gas sensors 35, where multiple sensors may provide added assurance that critical concentrations of gas 42 are accurately monitored. Gas sensor signal conditioner 36 may be a signal amplifier, where transmission from gas sensor 35 is amplified and routed through gas sensor signal conditioner 36. In one embodiment of the present invention, gas sensor signal conditioner 36 outputs gas percent or partial pressure output 39 to controller 14. Controller 14 may display information relative to gas concentrations in a visual display such as, for example, a user interface disclosed in U.S. patent application Ser. No. 60/330,853 filed Nov. 1, 2001, a data printout display, or in any other suitable means of informing user 13 of gas concentration. A further embodiment of the present invention comprises alerting user 13 of low gas concentration by a visual alarm, an audio alarm, or by other suitable alarms means.

Depending on sensor type, consumable components of an O₂ sensor may be gradually depleted by an oxidation reaction that is part of the measurement process. This oxidation reaction may continue even if solenoid-activated 2-way valve 44 is closed and O₂ sensor 35 is not in fluid communication or exposed to outflow gas 42. Continued oxidation is fueled by oxygen molecules trapped in a head space between solenoid-activated 2-way valve 44 and sensor 35 and helps to deplete the consumable components in an O₂ sensor. Therefore, to minimize continued oxidation from trapped O₂ molecules and to maximize sensor life, headspace accessible to an O₂ sensor within gas and monitoring system 9 may be designed to be as small as possible. Alternatively, if a small headspace is not practical in a particular embodiment, the present invention also contemplates evacuating a headspace between closed solenoid-activated 2-way valve 44 and O₂ sensor 35 via a pumping mechanism such as, for example, a vacuum pump (not shown) to remove trapped O₂ molecules and increase sensor life and/or replacement interval. Alternatively, oxygen may be purged from the headspace by flushing with an inert gas such as nitrogen. In yet another embodiment, the O₂ sensor may simply be left exposed to room air with no attempt made to minimize the number of trapped O₂ molecules during periods when 100% O₂ in outflow 42 is not being sampled.

Non-electrolyte-based O₂ sensors such as paramagnetic analyzers may also be preferentially supplied from an uninterruptible power supply or battery back-up in the event of main power supply failure, such that monitoring of O₂ supply is not discontinued.

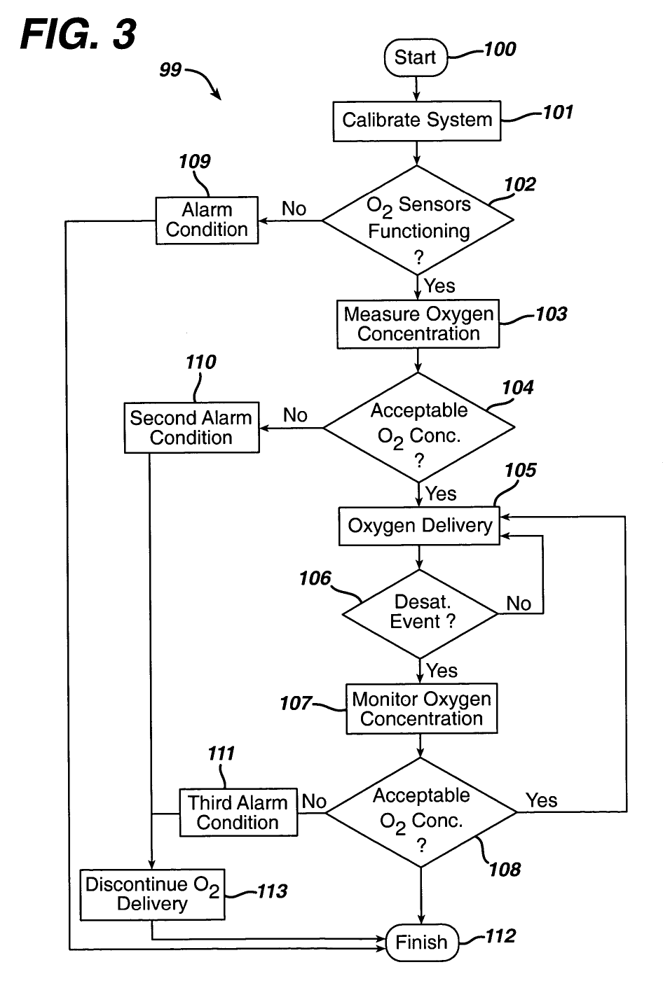

FIG. 3 illustrates one embodiment of a method for operating gas monitoring and delivery system 9, herein referred to as method 99. Start step 100 comprises activating gas monitoring and delivery system 9, where gas monitoring and delivery system 9 may be activated manually by user 13, from a remote location, automatically or contextually by controller 14, or by any other suitable activation means.

Step 101 of calibrating the system comprises, in one embodiment of the present invention, automatically calibrating gas sensor 35. Gas sensor 35 may be calibrated by taking a sample of room air, generally having an oxygen concentration of 21%, and evaluating output 39 to determine whether gas monitoring and delivery system 9 is indicating a proper oxygen concentration. A further means of calibrating gas sensor 35 comprises exposing gas sensor 35 to 100% oxygen, where a voltage output of gas sensor 35 is evaluated by controller 14 to determine what voltage corresponds to 100% oxygen concentration.

If a 2-point calibration is performed, two logical calibration mixtures are pure O₂ and room air because both are readily available. If only a one-point calibration is performed, the calibration may be either 100% O₂ or room air. Room air is preferentially used for a one-point calibration because it is always available as ambient air and it is safer for a patient if an O₂ analyzer reads accurately at 21% rather than at 100% O₂. For example, assume that there is 10% absolute error as a result of a one-point calibration, and further assume that % error increases linearly the further an actual gas mixture is from a calibration mixture. Thus, for a one-point calibration using 100% O₂, a 20% O₂ reading could be any value between 10% and 30% while a reading of 100% O₂ would be extremely accurate because 100% O₂ is the calibration point. Conversely, a reading of 90% O₂ for a one-point room air calibration could range from 80% to 100%. A 10% O₂ gas mixture inaccurately reading as a 20% O₂ mixture can have lethal consequences whereas a 90% O₂ gas mixture reading as 100% O₂ has less serious clinical consequences, if any. Additionally there is less chance of human error in room air composition than there is in the composition of 100% O₂ from a pipeline, cylinder, or calibration gas cylinder. Thus, room air may be considered as a more reliable calibration gas of known composition than 100% O₂ for the purposes of the invention.

Voltage output from gas sensor 35 will be evaluated as a function of a change in voltage output from a voltage corresponding to a concentration of 21% or 100% oxygen in determining a monitored concentration of oxygen throughout a procedure. For example, a galvanic cell may, when new, output 70 mV in the presence of 100% oxygen. Gas monitoring and delivery system 9 will then interpret a voltage output of 35 mV from gas sensor 35 as a concentration relative to an output voltage of gas sensor 35 in the presence of pure oxygen. As the galvanic cell deteriorates over time, it may output only 55 mV in the presence of pure oxygen. Gas monitoring and delivery system 9 will then associate an output of 55 mV from gas sensor 35 with a 100% oxygen concentration. In the scenario where the gas sensor outputs 55 mV in 100% oxygen, an output of 35 mV during a procedure will correspond to a higher concentration of oxygen in outflow gas 42 than it would for a gas sensor that outputs 70 mV in 100% oxygen. The present invention further comprises calibrating gas sensor 35 by other suitable means such as, for example, calibration with ambient air. A variety of gas sensors may be used in place of galvanic cells such as, for example paramagnetic sensors, in accordance with the present invention.

In one embodiment of the present invention, gas sensor 35 must exceed a predetermined voltage output in the presence of pure oxygen before gas monitoring and delivery system 9 will deliver oxygen. As gas sensor 35 decays over time, voltage output may decrease below acceptable levels when exposed to a calibration gas mixture. To verify proper gas sensor functionality, method 99 queries whether the oxygen sensors are functioning properly, herein referred to as query 102. In the event that the predetermined voltage output threshold is not exceeded by gas sensor 35, gas monitoring and delivery system 9 may initiate alarm condition 109.

Alarm condition 109 comprises alerting user 13 that gas sensor 35 is inoperative, where replacement of gas sensor 35 and, where applicable, a burn-in or warm-up period, is required before gas monitoring and delivery system 9 will activate gas delivery. Alarm condition 109 further comprises a visual alarm, an audio alarm, and/or other suitable alarms for indicating to user 13 that gas sensor 35 is inoperative. In the event that gas sensor 35 is functioning properly, method 99 will proceed to step 103 of measuring oxygen concentration.

Step 103 of measuring oxygen concentration comprises, in one embodiment of the present invention, measuring the concentration of oxygen in gas 42 before patient 18 receives gas 42. In order to improve patient safety, the present invention comprises determining whether gas 42 is the correct gas for an intended medical procedure. By determining the oxygen concentration of gas 42 and/or the concentration of critical gases such as, for example, nitrous oxide, method 99 guards against improper gas connections resulting in patient harm. Following step 103, method 99 will determine whether the measured oxygen concentration and/or concentration of critical gases of gas 42 corresponds to the appropriate gases and/or concentrations specified by user 13, herein referred to as query 104.

If query 104 results in an unacceptable oxygen concentration and/or an unacceptable critical gas concentration, method 99 may trigger second alarm condition 110. Second alarm condition 110 comprises alerting user 13 of insufficient oxygen and/or incorrect gas concentration of gas 42 via a visual alarm, an audio alarm, or by other suitable alarm means. Method 99 will then proceed to step 113 of discontinuing oxygen delivery and/or delivery of other gases associated with gas monitoring and delivery system 9. In the event that the oxygen concentration of gas 42 is acceptable and/or the correct gas is present, method 99 may proceed to step 105 of delivering O₂.

Step 105 of delivering oxygen comprises the delivery of oxygen, nitrous oxide, sedatives, analgesics, and/or other suitable gases, to patient 18. In one embodiment of the present invention, gas sensor 35 does not monitor the concentration of oxygen and/or other gases unless patient 18 experiences an oxygen desaturation event. While delivering oxygen and/or other gases, method 99 may query whether patient 18 has experienced a desaturation event, herein referred to as query 106. If a desaturation event does not occur, gas monitoring and delivery system 9 may continue to deliver oxygen and/or other gases in the absence of monitoring by gas sensor 35. If a desaturation event occurs, method 99 may proceed to step 107 of monitoring oxygen concentration. Monitoring oxygen and/or other gas concentrations at the beginning of a procedure and during potentially critical desaturation events, the present invention helps prolong the useful life of gas sensor 35 while improving patient safety. The present invention further comprises monitoring the concentration of oxygen and/or other gases at timed intervals or upon receipt of a manual command from user 13.

Following step 107, method 99 will proceed to query whether the monitored oxygen concentration and/or concentration of other critical gases is acceptable, herein referred to as query 108. If the concentration of gas 42 is hypoxic and/or contains an improper concentration of gases, method 99 may proceed to third alarm condition 111. Third alarm condition 111 comprises alerting user 13 via an audio alarm, a visual alarm, and/or any other suitable alarm means. Method 99 may further proceed to step 113 of discontinuing oxygen delivery including nitrous oxide, sedatives, analgesics, and/or other gases associated with gas monitoring and delivery system 9. If the oxygen concentration of gas 42 is acceptable following query 108, method 99 may proceed to step 105 of oxygen delivery.

The present invention comprises proceeding to finish step 112 following first alarm condition 109, second alarm condition 110, third alarm condition 111, and/or following a manual deactivation of gas monitoring and delivery system 9 by user 13.

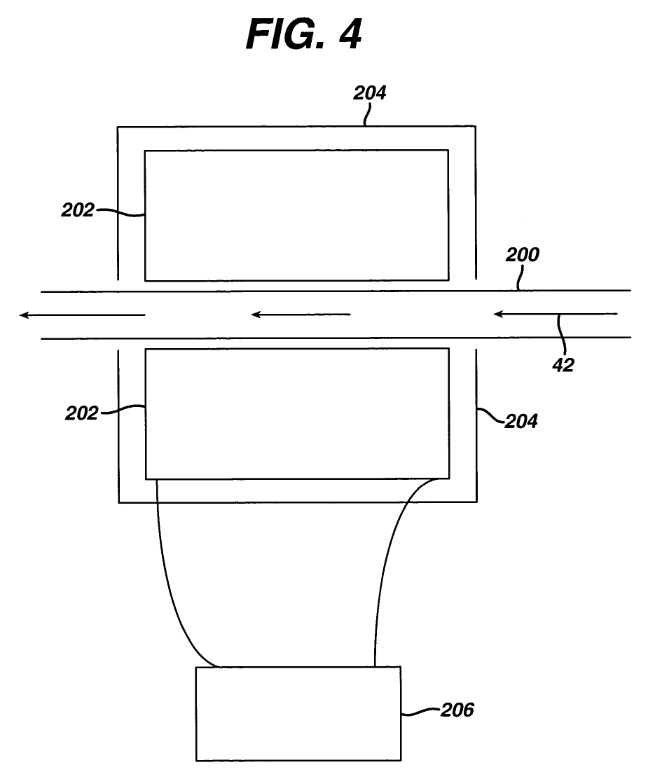

FIG. 4 depicts a gas conduit 200 for gas supplied to a gas delivery system. For example, conduit 200 may channel outflow gas 42 from gas monitoring and delivery system 9. Conduit 200 is fitted with an electrical coil 202 consisting of multiple turns of conductor, placed around gas conduit 200. Because of the paramagnetic properties of certain gas molecules, inductance of electrical coil 202 will change when a paramagnetic gas, such as oxygen, flows through gas conduit 200 placed inside the core of electrical coil 202, compared to when a non-paramagnetic gas such as N₂O flows through the conduit. Optionally, a cover 204 may be placed around electrical coil 202 to shield it from external influences such as magnetic and electric fields. The inductance of electrical coil 202 is processed by signal processing circuitry 206 to determine whether outflow gas 42 in gas conduit 200 is really O₂ or not. The binary O₂ sensor is applicable to all life support systems that deliver O₂, including, but not limited to medical systems, scuba systems, decontamination suits, high altitude breathing systems, astronaut breathing systems and fire rescue breathing systems.

A binary gas sensor may be implemented using any physical phenomena such as heat capacity, specific ratio, viscosity for which the gas of interest has unique or relatively unique or distinguishing properties. The paramagnetic property of oxygen was only meant as an example of implementing a binary gas sensor.

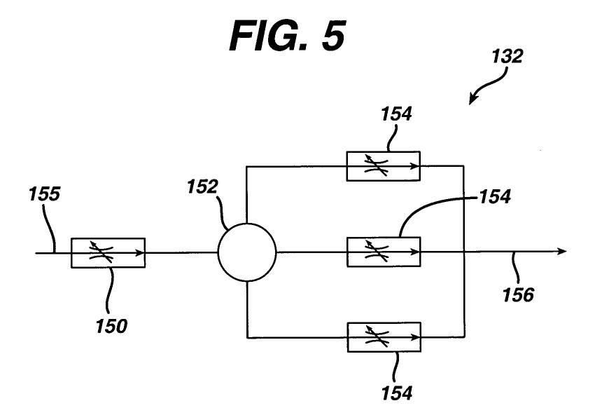

FIG. 5 illustrates an alternate embodiment of gas monitoring and delivery system 9 of the present invention where orifice assembly 132 can replace variable size orifice 32, which is shown in FIG. 2. Orifice assembly 132 further includes pressure regulator 150, N-Way valve 152, and discrete orifices 154. In this embodiment gas flows through pressure regulator 150 into N-Way Valve 152. Pressure regulator 150 helps control the pressure from input 155 to N-Way Valve 152 making any changes in supply pressure from input 155 negligible into N-Way valve 152. N-Way Valve 152 can be a valve that provides flow through only one discrete orifice 154 to output 156 or a valve that provides flow through several channels with orifices 154 providing a sum of flow to output 156. Orifice assembly 132 provides N discrete pressure levels where N is the number of discrete orifices 154. As shown in FIG. 5, N=3, thus, there is a 3-Way Valve which provides flow to the 3 discrete orifices 154 enabling 3 discrete pressure levels.

While the present invention has been illustrated by description of several embodiments, it is not the intention of the applicant to restrict or limit the spirit and scope of the appended claims to such detail. Numerous variations, changes, and substitutions will occur to those skilled in the art without departing from the scope of the invention. Moreover, the structure of each element associated with the present invention can be alternatively described as a means for providing the function performed by the element. Accordingly, it is intended that the invention be limited only by the spirit and scope of the appended claims.

What is claimed is:

1. A gas supply and monitoring system comprising:
a. a variable size orifice system connected to a gas source wherein said variable size orifice system has a gas outflow opposite of said gas source; and
b. a sensor system connected to said gas outflow wherein said sensor system can be used to verify a patient receives the appropriate gas from said gas source.

2. The gas supply and monitoring system of claim 1 wherein said gas source comprises of a gas containment dispenser.

3. The gas supply and monitoring system of claim 2 wherein said gas containment dispenser is an in-house gas supply.

4. The gas supply and monitoring system of claim 2 wherein said gas containment dispenser is a portable gas supply.

5. The gas supply and monitoring system of claim 2 wherein said gas containment dispenser comprises of oxygen.

6. The gas supply and monitoring system of claim 2 wherein said gas containment dispenser comprises of nitrous oxide.

7. The gas supply and monitoring system of claim 2 wherein said gas containment dispenser comprises of a sedative.

8. The gas supply and monitoring system of claim 2 wherein said gas containment dispenser comprises of an analgesic.

9. The gas supply and monitoring system of claim 1 wherein said variable size orifice system further comprises of a pressure relief valve and a control unit connected to a variable size orifice valve.

10. The gas supply and monitoring system of claim 9 wherein said variable size orifice system further comprises of a high side pressure sensor connected between said pressure relief valve and said variable size orifice valve and a low side pressure sensor connected to said gas outflow.

11. The gas supply and monitoring system of claim 1 wherein said variable size orifice system further comprises of and n-way valve connected to n discrete orifices wherein n is the number of pressure levels produced from said variable size orifice system.

12. The gas supply and monitoring system 1 wherein said sensor system further comprises gas sensor.

13. The gas supply and monitoring system 12 wherein said gas sensor is a galvanic cell.

14. A gas supply and monitoring system comprising:
a. a variable size orifice system connected to a gas source wherein said variable size orifice system has a gas outflow opposite of said gas source;
b. a sensor system connected to said gas outflow wherein said sensor system can be used to verify a patient receives the appropriate gas from said gas source; and
c. a control unit connected to said variable size orifice system and said sensor system wherein said control unit can be used to control the gas flow through said variable size orifice system and to control delivery of a sample of said gas outflow to said senor system.

15. The gas supply and monitoring system of claim 14 wherein said gas source comprises of a gas containment dispenser.

16. The gas supply and monitoring system of claim 15 wherein said gas containment dispenser is an in-house gas supply.

17. The gas supply and monitoring system of claim 15 wherein said gas containment dispenser is a portable gas supply.

18. The gas supply and monitoring system of claim 15 wherein said gas containment dispenser comprises of oxygen.

19. The gas supply and monitoring system of claim 15 wherein said gas containment dispenser comprises of nitrous oxide.

20. The gas supply and monitoring system of claim 15 wherein said gas containment dispenser comprises of a sedative.

21. The gas supply and monitoring system of claim 15 wherein said gas containment dispenser comprises of an analgesic.

22. The gas supply and monitoring system of claim 14 wherein said variable size orifice system further comprises of a pressure relief valve and a control unit connected to a variable size orifice valve.

23. The gas supply and monitoring system of claim 22 wherein said variable size orifice system further comprises of a high side pressure sensor connected between said pressure relief valve and said variable size orifice valve and a low side pressure sensor connected to said gas outflow.

24. The gas supply and monitoring system of claim 14 wherein said variable size orifice system further comprises of and n-way valve connected to n discrete orifices wherein n is the number of pressure levels produced from said variable size orifice system.

25. The gas supply and monitoring system of claim 14 wherein said sensor system further comprises gas sensor.

26. The gas supply and monitoring system of claim 25 wherein said gas sensor is a galvanic cell.

27. A method of delivering and monitoring a gas supply and monitoring system which comprises:
a. calibrating the gas supply and monitoring system;
b. verifying functionality of the sensors;
c. measuring gas concentration;
d. verifying the appropriate gas output and concentration level; and
e. delivering gas to the patient.

28. A method of delivering and monitoring a gas supply and monitoring system recited in claim 27 wherein checking said verifying functionality of the sensors further includes sounding an alarm if said sensors are not working.

29. A method of delivering and monitoring a gas supply and monitoring system recited in claim 27 wherein measuring gas concentration further includes sounding an alarm if said gas concentration is outside an acceptable range.

30. A method of delivering and monitoring a gas supply and monitoring system recited in claim 27 wherein monitoring gas concentration further includes sounding an alarm if said gas concentration is outside an acceptable range.
